(19) United States
(12) Patent Application Publication
(10) Pub. No.: US 2022/0328178 A1
(43) Pub. Date: Oct. 13, 2022

(54) SYSTEM AND METHOD FOR CORRELATING PULSE OXIMETRY WAVEFORM SIGNALS WITH BLOOD PRESSURE

(71) Applicant: Guy P. Curtis, San Diego, CA (US)

(72) Inventor: Guy P. Curtis, San Diego, CA (US)

(21) Appl. No.: 17/496,052

(22) Filed: Oct. 7, 2021

Related U.S. Application Data
(60) Provisional application No. 63/172,270, filed on Apr. 8, 2021.

(51) Int. Cl. G16H 40/63; A61B 5/0205; A61B 5/022; A61B 5/1455; A61B 5/00; A61B 5/1495; G16H 50/30

(52) U.S. Cl. CPC G16H 40/63; A61B 5/0205; A61B 5/02233; A61B 5/14552; A61B 5/7278; A61B 5/7425; A61B 5/7246; A61B 5/746; A61B 5/1495; G16H 50/30; A61B 5/0261

(57) ABSTRACT

A system for using an oximeter to provide blood pressure readings relies on a comparative interface between readings of a patient's blood flow waveform (oximeter) and blood pressure (sphygmomanometer) in his/her vasculature. For this purpose, a steady state condition is identified by calibrating a blood flow measurement A from the oximeter with a simultaneously obtained blood pressure measurement P from the sphygmomanometer. Further, using these simultaneous measurements, a blood pressure model is created that is based on the steady state. Thereafter, blood flow waveform readings from the oximeter are correlated with the steady state model to provide continuous blood pressure readings.

This application claims the benefit of U.S. Provisional Patent Application Ser. No. 63/172,270, filed Apr. 8, 2021. The entire contents of Application Ser. No. 63/172,270 are hereby incorporated by reference herein.

### FIELD OF THE INVENTION

The present invention pertains generally to blood pressure and blood flow monitoring systems. More particularly, the present invention pertains to systems and methods that continuously provide comprehensive information regarding the efficacy of a patient's heart muscle function. The present invention is particularly, but not exclusively, useful for periodically calibrating an oximeter with contemporaneous blood pressure measurements, taken by a sphygmomanometer, to continuously display information from the oximeter regarding a patient's heart rate, blood flow and blood pressure in a clinical environment.

### BACKGROUND OF THE INVENTION

An oximeter is a medical device that is well known for its ability to accurately indicate a patient's local blood flow characteristics. Specifically, an oximeter can record the sinusoidal characteristics of a blood flow waveform that provide both temporal and amplitude values. Of particular concern for the present invention are the magnitudes of sequential peak amplitudes and the time interval between these peak amplitudes in the blood flow waveform. With this information, a patient's heart rate and blood flow volume can be determined. These characteristics alone, however, do not indicate another important physical measurement, namely: blood pressure.

In a clinical environment it is important to have as much timely information as possible, for both a patient's blood flow and for his/her blood pressure. Collectively, this information is both interdependent and interrelated. However, unlike an oximeter which can be automatically operated continuously to record a blood flow waveform, the operation of a sphygmomanometer to measure blood pressure is labor intensive and can be realistically employed only intermittently. Heretofore, this operational disconnect has, for the most part, been tolerated.

A consequence of the interrelationship between the blood flow waveform and blood pressure is the fact there are three separately measurable characteristics of particular importance. These characteristics are variable and include: 1) the peak amplitude A of a pulse in the blood flow waveform; 2) the time interval Δt between these peak amplitudes (i.e., “heart rate”); and 3) blood pressure P. When considered together, collectively, these variables can lead to clinical conclusions that might otherwise have been missed.

With the above in mind, it is an object of the present invention to provide an oximeter which can be periodically calibrated, using a sphygmomanometer, to simultaneously provide continuous blood pressure readings with blood flow waveform information. Yet another object of the present invention is to incorporate heart rate information (±Δt) together with blood flow data (±ΔA) and blood pressure measurements (±ΔP) to provide a more comprehensive display presentation for clinical personnel with which to assess a patient's condition. Still another object of the present invention is to conduct continuous noninvasive blood pressure monitoring to elucidate new data concerning normal and abnormal states to greatly increase the diagnostic power and patient safety in the clinical environment. Another object of the present invention is to provide a system for continuously monitoring a patient's clinical condition which is easy to install, is simple to operate and which is comparatively cost effective.

### SUMMARY OF THE INVENTION

In accordance with the present invention, a system and method for continuously monitoring blood pressure in the vasculature of a patient requires a comparative interface between blood flow and blood pressure in the patient's vasculature. To do this, it is necessary to initially establish a steady-state condition for the relationship between a patient's blood flow measurement A and his/her blood pressure reading P. This steady-state condition is then used as input for a computer. In an operation of the system, the patient's blood flow waveform is continuously monitored, and continuously compared with a predetermined operational model. Based on this comparison, the blood flow waveform is used as a basis for displaying a corresponding blood pressure reading for the patient.

To establish a steady-state condition, a sphygmomanometer is used to obtain a blood pressure reading P_{measured}. As is common practice, P_{measured} defines the difference between a P_{systolic} pressure and a P_{diastolic} pressure. This blood pressure reading P_{measured} is then used to calibrate a blood flow measurement A_{calibrated}. More specifically, A_{calibrated} is concurrently obtained by an oximeter together with P_{measured} from a sphygmomanometer. Importantly, P_{measured} and A_{calibrated} are established simultaneously while the patient is in a steady state condition. The respectively identified P_{measured} and A_{calibrated} are then used as computer input to establish the patient's steady state condition for a computer operation.

After it has been calibrated, the oximeter is used to continuously monitor a local blood flow waveform of the patient. This waveform is typically sinusoidal, with each pulse in the waveform having a unique peak amplitude A. Also, the waveform will have a time interval Δt between the peak amplitude of each pulse and the peak amplitude of the immediately preceding pulse in the waveform. Thus, the waveform identifies a computer input that includes both a heart rate based on Δt and a blood flow volume based on both Δt and A. With this information the computer then employs a predetermined operational model that correlates changes in blood flow ±ΔA with changes in blood pressure ±ΔP.

In detail, for a constant blood flow condition, the predetermined operational model can be mathematically expressed as A=P/R, where R is a factor representing the patient's vascular resistance to blood flow. In this ratio relationship, changes in blood flow ±ΔA and changes in blood pressure ±ΔP are equated to each other for correlation purposes as ±ΔA/A_{calibrated}≈±ΔP/P_{measured}. Importantly, the correlation of blood flow with blood pressure in this operational model is made relative to the previously established steady-state condition. For this purpose the steady state condition can be expressed as (ΔP)_{base}=P_{systolic}−P_{diastolic}.

For the present invention, a display unit is provided for displaying blood pressure variations ±ΔP corresponding to variations in the blood flow waveform ±ΔA. As noted above, this correspondence is made in accordance with the operational model having a constant (ΔP)_{base}. Also, for the purpose of assessing blood flow in the patient's vasculature, the display unit will show whether there are any consequent changes in heart rate ±Δt that are associated with concurrently measured ±ΔA. Specifically, the display unit selectively presents ±ΔP in the context of either a first operational state wherein Δt is constant, or a second operational state wherein Δt is variable.

In the first operational state, Δt is constant and, to maintain a proper mathematically constant blood flow relationship, R is varied whenever A is varied in the operational model A=P/R. Specifically, with a +ΔA there will be a comparable change in R>1, and with a −ΔA there will be a comparable change in R<1. In the short term, this relationship can be considered valid because R is anatomically slow to vary. On the other hand, R may become a factor over a relatively longer term.

In the second operational state, Δt is variable, and R remains constant to maintain the operational relationship between ±ΔA and ±ΔP. Despite this fact, ±ΔA and ±ΔP may vary somewhat. In this latter case, recalibration may be necessary. Thus, in each operational state the operational model will determine a blood pressure P for display that may include important clinical information pertinent to the patient's condition.

Additional features for the present invention envision the use of a monitor for recording variations of ±ΔA and ±Δt in the blood flow waveform during a predetermined time duration. These measurements can then be compared with earlier measurements to determine whether ±ΔA and ±Δt have sufficiently stabilized during the predetermined time duration to identify a new value for the blood flow (e.g., A′). If so, the steady state condition should be recalibrated. For this purpose, the present invention envisions using a sphygmomanometer to periodically obtain new blood pressure readings P′_{measured} to recalibrate a new value for the blood flow A′ as A′_{calibrated} for use with P′_{measured} to identify a new steady state condition for the patient.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of the system for the present invention shown connected to a patient in a clinical environment;

FIG. 2 is graphical presentation of a patient's blood flow waveform;

FIG. 3 is a graphical presentation of a tracing profile for the patient's blood flow; and

FIG. 4 is a schematic presentation of the operational components for the present invention showing their interactive connections with each other for an operation of the present invention.

### DESCRIPTION OF THE PREFERRED EMBODIMENTS

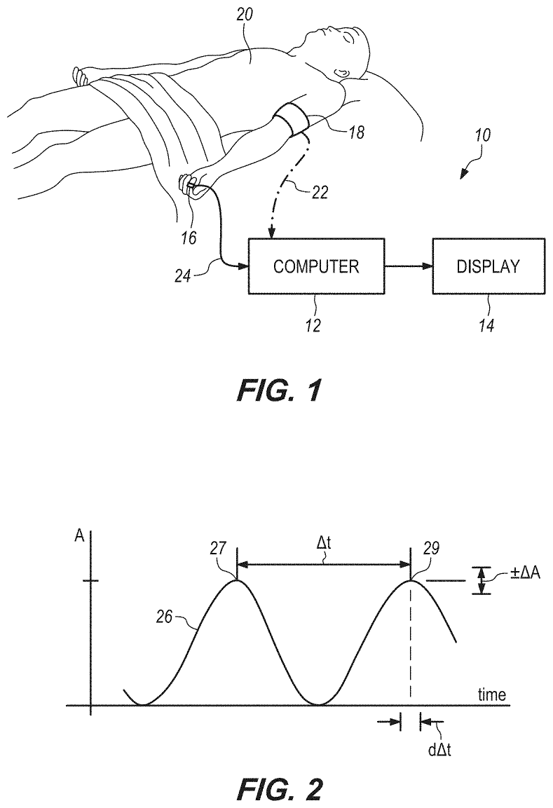

Referring initially to FIG. 1, a system for taking a blood pressure reading in accordance with the present invention is shown and is generally designated 10. As shown, the system 10 includes a computer 12 which is connected to a display 14. An oximeter 16 and a sphygmomanometer cuff 18 are typically placed on a patient 20 as shown in FIG. 1. The dot-dash line 22 connecting the sphygmomanometer cuff 18 with the computer 12 is shown differentiated to thereby indicate that this connection is not continuous. Instead, as is disclosed below, the sphygmomanometer cuff 18 is used primarily for the purpose of periodically calibrating the oximeter 16. On the other hand, the solid line 24 is shown to indicate a permanent connection between the oximeter 16 and the patient 20.

The purposes of the oximeter 16 and the sphygmomanometer cuff 18 are respectively of a type well known in the industry. Specifically, the sphygmomanometer cuff 18 is used to take periodic blood pressure readings, P, from the patient 20 that will include both a systolic pressure, P_{systolic}, and a diastolic pressure, P_{diastolic}. The oximeter 16, on the other hand, is used to identify a blood flow waveform 26 such as is shown in FIG. 2.

In FIG. 2 it is to be appreciated that all blood flow waveforms 26 are essentially sinusoidal and they have certain characteristics in common. Of these, the characteristics of particular interest for the present invention are changes in the magnitude ΔA of successive peaks in the waveform 26 and the time duration Δt between successive peaks. FIG. 2 shows these variable characteristics with reference to the peaks 27 and 29. Importantly, both variables (ΔA and Δt) are measured by the oximeter 16.

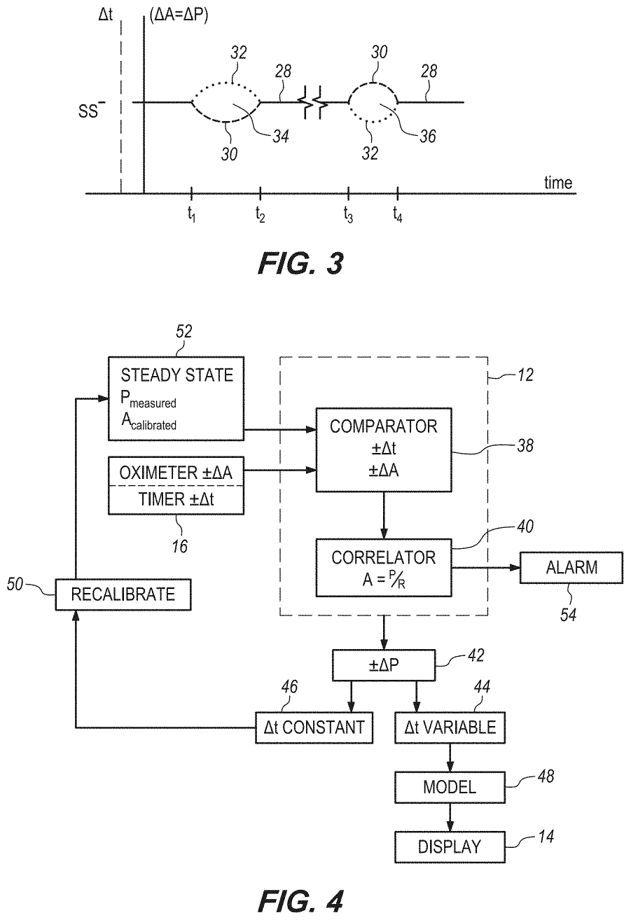

A consequence of changes in the respective magnitudes of ΔA and Δt is that as one increases the other typically decreases. As disclosed above, for purposes of the present invention, a steady state is established when the patient 20 is resting. With the patient 20 resting, a base relationship is created where A=P/R is constant, and R=1. In this base relationship (ΔA/sec)_{base}=(ΔP)_{base}=0. Nevertheless, when the patient 20 has a short-term episode with a ±ΔA the relationship ΔA=ΔP is still considered acceptable, at least in the short term. The consequence of this is best appreciated with reference to FIG. 3.

Examples for an operation of the present invention are provided below. In both of these examples, the steady state case is presented when R, in the expression A=P/R, is constant, with R=1. This situation is such that (ΔA/Δt)_{base}≈ΔP/R»±ΔP. Also, it must be appreciated that (ΔP)_{base} will be determined on a case-by-case basis and will vary accordingly.

By way of example, consider an operational model being established where (ΔP)_{base}=ΔP_{systolic}−ΔP_{diastolic}=120−60=60 and where, ΔP_{systolic} is approximated at ⅘ΔP, and ΔP_{diastolic} is approximated at ⅕ΔP. Moreover, it is important to recognize that when there is an increase in Δt there will be a drop in P. And vice-versa, when there is a decrease in Δt there will be a rise in P.

### Example 1 (Increase in Heart Rate Δt)

In accordance with the expression A=P/R, for an increase in heart rate by a factor of 2 (i.e., Δt decreases by ½), R in the expression A=P/R must also be considered equal to 2. Thus, ΔP=(ΔP)_{base}/2=30 for a decrease in ΔP. Thus, for this example, (ΔP)_{display} will equal [120−⅘(30)]/[60−⅕(30)] 96/54.

### Example 2 (Decrease in Heart Rate Δt)

In accordance with the expression A=P/R, for a decrease in heart rate by a factor of 15% (i.e., Δt increases by a factor of 1/0.85=1.18), R in the expression A=P/R must also be considered equal to 0.85. Thus, ΔP=(ΔP)_{base}/0.85=60/0.85=70.1) for an increase in ΔP. Thus, for this example, (ΔP)_{display} will equal [120+4/5(70.1)]/[60+1/5(70.1)]=176/74.

In FIG. 3, a tracing profile 28 is shown for the waveform 26. As indicated in FIG. 3, the tracing profile 28 has two different characteristic components, i.e., Δt and ΔA. These characteristic components are individually shown respectively in FIG. 3 as a dashed line 30 for Δt, and a dotted line 32 for ΔA. In a steady state (SS) condition for the waveform 26, i.e., when ΔA/Δt is considered to be constant, the tracing profile 28 is shown by a solid line. FIG. 3 also shows that in keeping with the expression A=P/R, ΔA=ΔP when these variables are in the steady state, SS.

Still referring to FIG. 3, examples for two different episodes of clinical interest for the present invention are shown. One is an increased pressure episode 34 for the patient 20, and the other is a decreased pressure episode 36 for the patient 20. During an increased pressure episode 34. Δt will decrease (i.e., heart rate increases) as indicated by dashed line 30. Concomitantly, the display 14 will show an increase in ΔP. On the other hand, during a decreased pressure episode 36, Δt will increase (i.e., heart rate will decrease) and ΔP will decrease. As recognized by the present invention, Δt and ΔA (a.k.a. ΔP) will both be measured by the oximeter 16.

With reference to FIG. 4, it will be seen that the computer 12 includes a comparator 38 and a correlator 40. As shown, the comparator 38 receives information from the oximeter 16 regarding both ±ΔA and ±Δt. These variables are then correlated by the correlator 40 to determine an output ±ΔP that is analyzed as indicated by the action block 42. Specifically, ±ΔP can be selectively analyzed relative to either a variable Δt at action block 44, or relative to a constant Δt (i.e., Δt=0) at action block 46.

In the circumstance when Δt is variable (block 44) the model 48 for P_{base} is evaluated as disclosed above. As also disclosed above, the result of this evaluation is shown on the display 14. On the other hand, when Δt is considered a constant (block 46) the variable R is evaluated for a possible recalibration (block 50) of the expression A=P/R or for sounding an alarm 54. In the event recalibration is implemented, the steady state SS for P_{measured} and A_{calibrated} with a new (ΔP)_{base} is established using the oximeter 16 and the sphygmomanometer 18. This action is indicated by action block 52.

While the particular System and Method for Correlating Pulse Oximetry Waveform Signals with Blood Pressure as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for continuously monitoring blood pressure in the vasculature of a patient which comprises:
an oximeter positioned on the patient to measure a sinusoidal waveform representing a patient's local blood flow having a peak amplitude for each pulse in the waveform, and a time interval Δt between the peak amplitudes of sequential pulses in the waveform, to collectively identify a blood flow A,
a sphygmomanometer for obtaining a blood pressure reading P for the patient, wherein P is defined as a difference between a P_{systollic} pressure and a P_{diastolic} pressure;
a computer for receiving a blood flow measurement A from the oximeter which is calibrated with a simultaneously obtained blood pressure reading P from the sphygmomanometer, to respectively identify an A_{calibrated} and a P_{measured} for use as computer input for a patient's steady state condition, wherein the computer employs an operational relationship expressed as A=P/R wherein R is a factor representing a vascular resistance to the patient's blood flow A, to correlate changes in blood flow ±ΔA with changes in blood pressure ±ΔP relative to the steady state condition of the patient; and
a display unit for displaying ±ΔP based on ±ΔA, and displaying whether there is any consequent ±Δt associated with the measured ±ΔA for assessing blood flow in the patient's vasculature.

2. The system of claim 1 wherein the computer employs a ratio relationship between changes in blood flow ±ΔA and changes in blood pressure ±ΔP, expressed as ±ΔA/A_{calibrated}≈±ΔP/P_{measured} for correlation purposes.

3. The system of claim 2 wherein the display unit selectively presents ±ΔP in the context of:
a first operational state when Δt is constant, and R is variable to maintain the operational relationship A=P/R, with R>1 for a +ΔA and R<1 for a −ΔA; and
a second operational state when Δt is variable, and R is constant to maintain the operational relationship between ±ΔA and ±ΔP with R=1.

4. The system of claim 3 wherein ΔP=ΔP_{systolic}−ΔP_{diastolic}, wherein ΔP_{systolic} is approximated as being 4/5ΔP, and wherein ΔP_{diastolic} is approximated as being 1/5ΔP.

5. The system of claim 4 wherein the display unit presents P as a P_{systolic}=P_{measured}+ΔP_{systolic}, and a P_{diastolic}=P_{measured}±ΔP_{diastolic}.

6. The system of claim 4 wherein the computer comprises:
a timer for measuring Δt between sequential pulses in the blood flow waveform;
a comparator for comparing a preceding Δt with the peak amplitude of the immediately following pulse in the blood flow A; and
a correlator for analyzing Δt, together with ±ΔA for each pulse, to identify, for display, the operational state of blood flow A in the patient.

7. The system of claim 6 further comprising an alarm connected to the correlator of the computer to alert clinical personnel of a significant change in the blood flow and blood pressure condition.

8. The system of claim 4 wherein a steady state condition for the patient is periodically recalibrated in accordance with clinical requirements.

9. The system of claim 6 wherein the comparator further comprises a monitor for recording variations ±ΔA and ±Δt of the blood flow waveform during a predetermined period of time, to determine whether ±ΔA and ±Δt have sufficiently stabilized during the predetermined time period to identify a new value for the blood flow A′.

10. The system of claim 9 wherein the sphygmomanometer obtains a new blood pressure reading P′_{measured} to recalibrate a new value for the blood flow A′ as A′_{calibrated} for use with P′_{measured} to identify the patient's steady state condition.

11. A system for continuously monitoring blood flow characteristics in the vasculature of a patient which comprises:
a means for monitoring a local blood flow waveform of the patient, wherein the waveform is sinusoidal and each pulse in the waveform has a peak amplitude A with a time interval Δt between the peak amplitude of the pulse and the peak amplitude of the immediately preceding pulse in the waveform, to collectively identify a heart rate based on Δt and a blood flow volume based on both Δt and A;
a means for calibrating A with a measured blood pressure reading P_{measured} for a sequence of pulses in the waveform, to identify an A_{calibrated}, wherein P_{measured} and A_{calibrated} are established simultaneously while the patient is in a steady state condition; and
a computer for continuously receiving data from the monitoring means pertinent to variations in heart rate ±Δt and variations in peak amplitudes ±ΔA, to identify ±ΔA corresponding to changes in blood pressure ±ΔP based on a predetermined operational relationship which correlates changes in peak amplitudes ±ΔA as changes in blood pressure ±ΔP relative to the steady state of the patient, and further wherein variations in heart rate ±Δt are evaluated in context with ±ΔA for assessing blood flow in the patient's vasculature.

12. The system of claim 11 wherein the monitoring means is an oximeter and the calibrating means is a sphygmomanometer.

13. The system of claim 11 wherein the predetermined operational relationship is expressed as A=P/R where R is a factor representing a vascular resistance to the patient's blood flow, and wherein ±ΔP is considered in the context of:
a first operational state when Δt is constant, and R is variable to maintain the operational relationship A=P/R, wherein with R>1 there is a +ΔA, and with R<1 there is a −ΔA; and
a second operational state when Δt is variable, and R is constant to maintain the operational relationship between ±ΔA and ±ΔP with R=1.

14. The system of claim 13 further comprising a display unit, wherein the display unit presents P as a P_{systolic}=P_{measured}±ΔP_{systolic}, and a P_{diastolic}=P_{measured}±ΔP_{diastolic}, and further wherein ΔP=ΔP_{systolic}−ΔP_{diastolic}, wherein ΔP_{systolic} is approximated as being 4/5ΔP, and wherein ΔP_{diastolic} is approximated as being 1/5ΔP.

15. The system of claim 14 wherein variations ±ΔA and ±Δt of the blood flow waveform are monitored during a predetermined period of time, to determine whether ±ΔA and ±Δt have sufficiently stabilized during the predetermined time period to identify a new value for the blood flow A′, and wherein thereafter the sphygmomanometer obtains a new blood pressure reading P′_{measured} corresponding to A′ to recalibrate a new value for the blood flow A as A′_{calibrated} for use with P′_{measured} to identify the patient's steady state condition.

16. A method for calibrating an oximeter to provide continuous blood pressure and heart function information from a patient in a clinical environment, which comprises the steps of:
creating an environment wherein the patient's heart muscle is stabilized to represent a steady state condition with a blood flow A;
obtaining a blood pressure reading P measured for the patient during the steady state condition, wherein P is defined as a difference between a systolic pressure P_{systolic} and a diastolic pressure P_{diastolic}, and P is designated P_{measured};
calibrating the patient's blood flow A with P_{measured} to establish a calibrated value A_{calibrated} for the patient's blood flow;
providing P_{measured} and A_{calibrated} as input for a computer;
monitoring an output from the oximeter at the computer to equate changes in blood flow ±ΔA relative to A_{calibrated} with changes in blood pressure ±ΔP relative to P_{measured} based on a ratio relationship where ±ΔA/A_{calibrated}=±ΔP/P_{measured}, and wherein the computer further employs an operational relationship expressed as A=P/R where R is a factor representing a vascular resistance to the patient's blood flow A, to correlate changes in blood flow ±ΔA with changes in blood pressure ±ΔP relative to the steady state condition of the patient; and
displaying P in the clinical environment as a P_{systolic}=P_{measured}±ΔP_{systolic}, and a P_{diastolic}=P_{measured}±ΔP_{diastolic}.

17. The method of claim 16 wherein A has a sinusoidal waveform representing a patient's local blood flow with a peak amplitude for each pulse in the waveform, and a time interval Δt between the peak amplitudes of sequential pulses in the waveform, to collectively identify the blood flow A.

18. The method of claim 17 wherein the displaying step further comprising the steps of:
evaluating whether there is any consequent ±Δt associated with the measured ±ΔA for assessing blood flow in the patient's vasculature;
presenting a first operational state when Δt is constant, and R is variable to maintain the operational relationship A=P/R, wherein with R>1 there is a +ΔA, and with R<1 there is a −ΔA; and
presenting a second operational state when Δt is variable, and R is constant to maintain the operational relationship between ±ΔA and ±ΔP with R=1.

19. The method of claim 18 further comprising the steps of:
recording variations ±ΔA and ±Δt of the blood flow waveform during a predetermined period of time, to determine whether ±ΔA and ±Δt have sufficiently stabilized during the predetermined time period to identify a new value for the blood flow A′; and
obtaining a new blood pressure reading P′_{measured} to recalibrate a new value for the blood flow A′_{calibrated} for use with P′_{measured} to identify the patient's steady state condition.

20. The method of claim 17 wherein the steady state condition for the patient is periodically recalibrated in accordance with clinical requirements.
